(19) United States
(12) Patent Application Publication
(10) Pub. No.: US 2004/0191204 A1
(43) Pub. Date: Sep. 30, 2004

(54) Composition for washing keratinous substances comprising at least one alkyl ether sulphate and at least one amphoteric surfactant

(76) Inventors: Jonathan Gawtrey, Boulogne (FR); Mireille Maubru, Chatou (FR)

Correspondence Address: Thomas L. Irving, FINNEGAN, HENDERSON, FARABOW, GARRETT & DUNNER, L.L.P., 1300 I Street, N.W., Washington, DC, 20005-3315, US

(21) Appl. No.: 10/739,118

(22) Filed: Dec. 19, 2003

Related U.S. Application Data
(60) Provisional application No. 60/439,451, filed on Jan. 13, 2003.

(30) Foreign Application Priority Data
Dec. 19, 2002 (FR) 02 16195

(51) Int. Cl. A61K 7/075; A61K 7/08

(52) U.S. Cl. 424/70.21

(57) ABSTRACT

The present invention relates to compositions for washing keratinous substances, for example hair and/or the skin, comprising at least one alkyl ether sulphate anionic surfactant and at least 2.5% of at least one alkylamphohydroxyalkylsulphonate amphoteric surfactant, the weight ratio of the at least one alkyl ether sulphate surfactant to the at least one alkylamphohydroxyalkylsulphonate amphoteric surfactant being greater than or equal to 1, and to the washing process employing these compositions.

This application claims benefit of U.S. Provisional Application No. 60/439,451, filed Jan. 13, 2003.

The present invention relates to compositions for washing keratinous substances, for example hair and/or the skin, comprising at least one alkyl ether sulphate anionic surfactant and at least one alkylamphohydroxyalkylsulphonate amphoteric surfactant and to a washing process employing these compositions.

The use of amphoteric surfactants, such as alkylimidazolines or betaines, in shampoos is known. These surfactants act as additional detergents in this type of formulation.

Alkylamphohydroxyalkylsulphonate amphoteric surfactants have already been recommended in detergent cosmetic compositions. They were disclosed, for example, in Patent Application WO 99/36054.

However, these compositions are not entirely satisfactory, for example, they do not exhibit satisfactory cosmetic properties, such as the disentangling and the smoothing of dried hair.

The inventors have discovered, surprisingly, that it is possible to formulate compositions for washing keratinous substances, such as shampoos, by using at least one amphoteric surfactant in combination with at least one alkyl ether sulphate surfactant.

Disclosed herein is a composition for washing keratinous substances comprising at least one alkyl ether sulphate anionic surfactant and at least 2.5% by weight of at least one alkylamphohydroxyalkylsulphonate amphoteric surfactant, wherein the weight ratio of the at least one alkyl ether sulphate anionic surfactant to the at least one alkylamphohydroxyalkylsulphonate amphoteric surfactant is greater than or equal to 1, for example ranging from 2 to 20, from 2 to 10, and from 3 to 7.

Also disclosed herein is a method of improving at least one of, for example, the disentangling and the smoothing of keratinous substances, comprising applying to keratinous substances a composition comprising at least 2.5% by weight of at least one alkylamphohydroxyalkylsulphonate amphoteric surfactant and at least one alkyl ether sulphate anionic surfactant.

Also disclosed herein is a process for washing keratinous substances.

Other aspects of the invention will become apparent on reading the description and examples which follow. The various aspects of the invention will now be described in detail. All the meanings and definitions of the compounds used in the disclosed invention given below are valid for all aspects of the invention.

As used herein, the term “keratinous substances” is understood to mean the hair, eyelashes, eyebrows, skin, nails, mucous membranes or scalp, and for example the hair.

The at least one alkylamphohydroxyalkylsulphonate amphoteric surfactant may have the following formula (I):

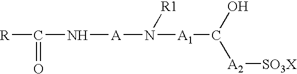

wherein:

R is chosen from saturated and unsaturated, linear and branched, hydrocarbonaceous radicals comprising from 5 to 29 carbon atoms, for example, R is chosen from alkyl and mono- and polyunsaturated alkenyl radicals comprising from 5 to 29 carbon atoms, and as a further example from 7 to 22 carbon atoms, and as an additional example from 9 to 17 carbon atoms,

R1 is a C₁-C₄ hydroxyalkyl radical, for example a hydroxyethyl radical, A, A₁ and A₂, which are identical or different, are chosen from linear and branched, divalent, C₁-C₁₀, alkylene radicals, for example C₁-C₃, alkylene radicals, and

X is chosen from a hydrogen atom, an inorganic cation, and an organic cation, such as: that of an alkali metal (for example, Na⁺ or K⁺), that of an alkaline earth metal (Mg²⁺, Ca²⁺), the NH₄⁺ ion or the ammonium ions resulting from basic amino acids or from aminoalcohols.

Examples of compounds for use in the disclosed invention include compounds of formula (I) wherein R for example is chosen from saturated, linear and branched, alkyl radicals comprising from 7 to 29 carbon atoms, for example from 7 to 22 carbon atoms.

When X is an ammonium ion resulting from an alkanolamine, the latter can be chosen from monoethanolamine, diethanolamine, triethanolamine, and 3-amino-1,2-propanediol. When X is an ammonium ion resulting from an amine, this amine can be a basic amino acid, such as lysine, arginine, sarcosine, ornithine or citrulline.

In an embodiment, A=A₂ and is —CH₂CH₂—, A₁ is —CH₂—, and X is Na^{+.}

Mention may for example be made, among the surfactants of formula (I), of cocoylamphohydroxypropylsulphonate salts and for example the sodium salt, such as the product provided under the name MIRANOL CSE by Rhodia Chimie, or palmitoylamphohydroxypropylsulphonate salts and for example the sodium salt, such as the product provided under the name MIRANOL CS by Rhodia Chimie.

In an embodiment, the at least one amphoteric surfactant chosen from the alkylamphohydroxyalkylsulphonate compounds and their salts may be present in the composition in an amount ranging from 2.5% to 30% by weight, for example from 3% to 15% by weight with respect to the total weight of the final composition.

The at least one alkyl ether sulphate anionic surfactant which may be used, alone or as mixtures, in the disclosed invention are the salts (for example alkali metal salts, such as sodium salts, ammonium salts, amine salts, aminoalcohol salts or magnesium salts) of alkyl ether sulphates, alkylamido ether sulphates or alkylaryl ether sulphates. The alkyl radical of all these various compounds comprises, for example, from 8 to 24 carbon atoms and the aryl radical for example is chosen from a phenyl group and a benzyl group.

The mean number of ethylene oxide or propylene oxide groups may range for example from 2 to 50, and as a further example from 2 to 10.

In an embodiment, these anionic surfactants may be the salts of C₈-C₁₄ alkyl ether sulphates, and for example those of C₁₂-C₁₄ alkyl ether sulphates. These salts for example comprise from 2 to 5 ethylene oxide groups.

The at least one alkyl ether sulphate anionic surfactant may be present in an amount ranging from 2.5% to 50% by weight, for example from 5 to 20% by weight, with respect to the total weight of the composition.

The weight ratio of the at least one alkyl ether sulphate surfactant to the at least one amphoteric surfactant is greater than or equal to 1, for example ranging from 2 to 20, from 2 to 10, and from 3 to 7.

The composition disclosed herein further comprises at least one surfactant chosen from nonionic, amphoteric or zwitterionic surfactants other than the at least one amphoteric surfactant defined above.

The at least one surfactant other than the at least one amphoteric surfactant described above may be present in an amount ranging from 0.5 to 15% by weight with respect to the total weight of the composition.

The total amount of surfactants in the composition disclosed herein ranges from 5 to 55% by weight, and for example from 7 to 30% by weight, with respect to the total weight of the composition.

The at least one nonionic surfactant may be chosen from, for example, polyethoxylated, polypropoxylated fatty alcohols, polyglycerolated fatty alcohols, alkylphenols, acids with a fatty chain comprising from 8 to 20 carbon atoms, the number of ethylene oxide or propylene oxide groups ranging from 2 to 50, and the number of glycerol groups ranging from 2 to 30, and oxyethylenated C₈-C₃₀ fatty acid sorbitan esters with a number of moles of ethylene oxide ranging from 2 to 30.

Mention may also be made of copolymers of ethylene oxide and of propylene oxide; condensates of ethylene oxide and of propylene oxide with fatty alcohols; polyethoxylated fatty amides for example having 2 to 30 mol of ethylene oxide; polyglycerolated fatty amides for example comprising 1 to 5 glycerol groups and for example 1.5 to 4; sorbitan fatty acid esters oxyethylenated with 12 to 30 mol of ethylene oxide; sucrose fatty acid esters, polyethylene glycol fatty acid esters, alkylpolyglycosides, carbamate or amide derivatives of N-alkylglucamines, aldobionamides, or amine oxides, such as alkylamine oxides or N-(acylamidopropyl)morpholine oxides.

The composition may also comprise at least one cationic surfactant for example present in an amount ranging from 0.001 to 5% by weight, with respect to the total weight of the composition.

The at least one cationic surfactant may be chosen for example from optionally polyoxyalkylenated, primary, secondary or tertiary fatty amine salts; quaternary ammonium salts; imidazoline derivatives; and amino oxides possessing a cationic nature.

Examples of quaternary ammonium salts are tetraalkylammonium halides (for example chlorides), such as, for example, dialkyldimethylammonium or alkyltrimethylammonium chlorides wherein the alkyl radical comprises approximately from 12 to 22 carbon atoms, such as behenyltrimethylammonium chloride, distearyldimethylammonium chloride, cetyltrimethylammonium chloride, benzyldimethylstearylammonium chloride or stearamidopropyidimethyl(myristyl acetate)ammonium chloride, sold under the name of CEPHARYL 70 by Van Dyk.

Mention may also be made of di(acyloxyethyl)dimethylammonium, diacyloxyethyl(hydroxyethyl)methylammonium, mono(acyloxyethyl)di(hydroxyethyl )methylammonium, tri(acyloxyethyl)methylammonium and monoacyloxyethyl(hydroxyethyl)dimethylammonium salt (such as chlorides or methyl sulphates) and their mixtures.

The acyl radicals originate for example from a vegetable oil, such as palm oil or sunflower oil.

The amount of the at least one surfactant other than the at least one anionic surfactant present in the composition may vary from 0 to 30%, and for example from 0.5 to 15% by weight with respect to the total weight of the composition.

The composition disclosed herein may also comprise at least one silicone, such as polyorganosiloxane oils or polyorganosiloxane gums or resins, such as in the form of solutions in organic solvents or in the form of emulsions or microemulsions.

Mention may be made, among the polyorganosiloxanes which may be used in the disclosed composition, without implied limitation, of:

volatile silicones: those having a boiling point ranging from 60° C. to 260° C. They are chosen from cyclic silicones comprising from 3 to 7 silicon atoms and for example 4 to 5; and

nonvolatile silicones: they are composed mainly of polyalkylsiloxanes, polyarylsiloxanes, polyalkylarylsiloxanes, silicone gums, silicone resins or their mixtures and organomodified silicones.

Use may for example be made of polydimethylsiloxanes, aminated silicones, and oxyalkylenated silicones.

The at least one silicone (polyorganosiloxanes) may be used in the composition of the invention amounts ranging from 0.01 to 20% by weight and for example from 0.1 to 10% by weight, with respect to the total weight of the composition.

The composition of the invention may also comprise at least one cationic or amphoteric polymer.

The at least one cationic polymer may have a number-average molecular mass ranging from 500 to 5×10⁶ approximately, and for example ranges from 10³ to 3×10⁶ approximately.

Mention may be made, among the at least one cationic polymer, of quaternized proteins (or protein hydrolysates) and polymers of the polyamine, polyaminoamide and poly(quaternary ammonium) type. These are known products.

The quaternized proteins or protein hydrolysates may be, for example, chemically modified polypeptides carrying quaternary ammonium groups at the chain end or grafted to the chains. Their molecular mass may vary, for example, from 1,500 to 10,000, and as a further example from 2,000 to 5,000 approximately.

The polymers of the polyamine, polyaminoamide or poly(quaternary ammonium) type which may be used in accordance with the present invention which may be mentioned, for example, are those disclosed in French Patents No. 2 505 348 or 2 542 997, the disclosures of both of which are hereby incorporated by reference. Mention may be made, among these polymers, of:

(1) Optionally quaternized vinylpyrrolidone/dialkylaminoalkyl acrylate or methacrylate copolymers, such as the polymers disclosed in detail in French Patents 2 077 143 and 2 393 573, the disclosures of both of which are hereby incorporated by reference.

(2) The cellulose ether derivatives comprising quaternary ammonium groups disclosed in French Patent 1 492 597, the disclosure of which is hereby incorporated by reference.

(3) Cationic cellulose derivatives, such as the copolymers of cellulose or the cellulose derivatives grafted with a water-soluble quaternary ammonium monomer and disclosed in particular in U.S. Pat. No. 4,131,576, the disclosure of which is hereby incorporated by reference, such as hydroxyalkyl celluloses, for example hydroxymethyl, hydroxyethyl or hydroxypropyl celluloses, grafted for example with a methacryloylethyltrimethylammonium, methacrylamidopropyltrimethylammonium or diallyldimethylammonium salt.

(4) Non-cellulose polysaccharides, and for example cationic guar gums, disclosed for example in U.S. Pat. Nos. 3,589,578 and 4,031,307, the disclosures of both of which are hereby incorporated by reference.

(5) Polymers composed of piperazinyl units and of divalent, straight- or branched-chain alkylene or hydroxyalkylene radicals, optionally interrupted by oxygen, sulphur or nitrogen atoms or by aromatic or heterocyclic rings, as well as the oxidation and/or quaternization products of these polymers. Such polymers are disclosed for example in French Patents 2 162 025 and 2 280 361, the disclosures of both of which are hereby incorporated by reference.

(6) Water-soluble polyaminoamides prepared for example by polycondensation of an acidic compound with a polyamine. These polyaminoamides may be crosslinked by an epihalohydrin, a diepoxide, a dianhydride, an unsaturated dianhydride, a bisunsaturated derivative, a bishalohydrin, a bisazetidinium, a bishaloacyldiamine or an alkyl bishalide or alternatively by an oligomer resulting from the reaction of a bifunctional compound reactive with respect to a bishalohydrin, a bisazetidinium, a bishaloacyldiamine, an alkyl bishalide, an epihalohydrin, a diepoxide or a bisunsaturated derivative. The crosslinking agent may be used in amounts ranging from 0.025 to 0.35 mol per amine group of the polyaminoamide. These polyaminoamides may be alkylated or, if they comprise at least one tertiary amine functional group, they may be quaternized. Such polymers are disclosed in particular in French Patents 2 252 840 and 2 368 508, the disclosures of both of which are hereby incorporated by reference.

(7) Polyaminoamide derivatives resulting from the condensation of polyalkylenepolyamines with polycarboxylic acids, followed by an alkylation by bifunctional agents. Mention may be made, for example, of adipic acid/dialkylaminohydroxyalkyldialkylenetriamine polymers wherein the alkyl radical comprises from 1 to 4 carbon atoms, and for example is chosen from methyl, ethyl, and propyl groups. Such polymers are disclosed, for example, in French Patent 1 583 363, the disclosure of which is hereby incorporated by reference.

(8) Polymers obtained by reaction of a polyalkylenepolyamine comprising two primary amine groups and at least one secondary amine group with a dicarboxylic acid chosen from diglycolic acid and saturated aliphatic dicarboxylic acids having from 3 to 8 carbon atoms, the molar ratio of polyalkylenepolyamine to dicarboxylic acid ranging from 0.8:1 to 1.4:1 and the polyaminoamide resulting therefrom being reacted with epichlorohydrin in a molar ratio of epichlorohydrin in relation to the secondary amine group of the polyaminoamide ranging from 0.5:1 to 1.8:1. Such polymers are disclosed, for example, in U.S. Pat. Nos. 3,227,615 and 2,961,347, the disclosures of both of which are hereby incorporated by reference.

(9) Cyclopolymers of methyldiallylamine or of diallyldimethylammonium, such as those disclosed in French Patent 2 080 759 and in its Certificate of Addition 2 190 406, the disclosures of both of which are hereby incorporated by reference.

(10) The di(quaternary ammonium) polymers disclosed in French Patents 2 320 330, 2 270 846, 2 316 271, 2 336 434, and 2 413 907 and U.S. Pat. Nos. 2,273,780; 2,375,853; 2,388,614; 2,454,547; 3,206,462; 2,261,002; 2,271,378; 3,874,870; 4,001,432; 3,929,990; 3,966,904; 4,005,193; 4,025,617; 4,025,627; 4,025,653; 4,026,945; and 4,027,020, the disclosures of all of which are hereby incorporated by reference.

(11) Poly(quaternary ammonium) polymers, such as disclosed in Patent Application EP-A-122 324, the disclosure of which is hereby incorporated by reference.

(12) Homopolymers or copolymers derived from acrylic or methacrylic acids and comprising CH₂—CHRₐ—CO—O—A₁—NRₑR_{f}, CH₂—CHRₐ—CO—O—A₁—N₊R_{b}R_{c}R_{d}X⁻ and/or CH₂—CHRₐ—CO—NH—A₁—N⁺R_{b}R_{c}R_{d}X⁻ units wherein the Rₐ groups independently are chosen from H and CH₃, the A₁ groups independently are chosen from linear and branched alkylene group comprising from 1 to 6 carbon atoms and a hydroxyalkylene group of 1 to 4 carbon atoms, the R_{b}, R_{c} and R_{d} groups, which are identical or different, independently are chosen from an alkyl group having 1 to 18 carbon atoms and a benzyl radical, the Rₑ and R_{f} groups, which are identical or different, are chosen from a hydrogen atom and an alkyl group of 1 to 6 carbon atoms, and X⁻ is an anion, for example methyl sulphate or halide, such as chloride or bromide.

(13) Quaternary polymers of vinylpyrrolidone and of vinylimidazole, such as, for example, the products sold under the names LUVIQUAT FC 905, LUVIQUAT FC 550 and LUVIQUAT FC 370 by BASF.

(14) Polyamines, such as POLYQUART H sold by Henkel, referenced under the name “Polyethylene Glycol Tallow Polyamine” in the CTFA dictionary.

(15) Crosslinked polymers of methacryloyloxyethyltrimethylammonium salt, such as the polymers obtained by homopolymerization of dimethylaminoethyl methacrylate quaternized by methyl chloride or by copolymerisation of acrylamide with dimethylaminoethyl methacrylate quaternized by methyl chloride. The homo- or copolymerisation being followed by a crosslinking by a compound possessing olefinic unsaturation, such as methylenebisacrylamide. Use may, for example, be made of a crosslinked acrylamide/methacryloyloxyethyltrimethylammonium chloride (20/80 by weight) copolymer in the form of a dispersion comprising 50% by weight of the copolymer in mineral oil.

Other cationic polymers which may be used in the disclosed invention are polyalkyleneimines, such as polyethyleneimines, polymers comprising vinylpyridine or vinylpyridinium units, condensates of polyamines and of epichlorohydrin, quaternary polyureylenes and chitin derivatives.

In an embodiment, the cationic polymers capable of being used in the disclosed invention include cellulose ether derivatives comprising quaternary ammonium groups, non-cellulose polysaccharides, and for example cationic guar gums, and cyclopolymers of methyldiallylamine or of diallyldimethylammonium.

The cationic polymers may be used in the disclosed composition in an amount ranging from 0.001 to 20% by weight and for example from 0.01 to 5% by weight, with respect to the total weight of the composition.

The disclosed composition may exhibit a pH generally ranging from 3 to 12, and for example from 4 to 8.

The cosmetically acceptable medium of the disclosed compositions may be composed either of water or of a mixture of water and of at least one solvent chosen from lower alcohols, alkylene glycols and polyol ethers.

The disclosed composition may also comprise viscosity-regulating agents, such as electrolytes, for example sodium chloride, or thickeners, for example cellulose derivatives, such as, for example, carboxymethylcellulose, hydroxypropylcellulose or hydroxyethylcellulose, guar gum, hydroxypropylated guar gums, scleroglucans, xanthan gum or amphiphilic polymers comprising at least one fatty chain.

The disclosed composition may also comprise various adjuvants generally used in cosmetics, such as fragrances, preservatives, foam synergists, sequestering agents, foam stabilizers, propellants, colorants, antidandruff agents, ceramides, vitamins or provitamins, hydroxy acids, acidifying or basifying agents, vegetable, mineral or animal oils, synthetic oils, such as polyisobutenes and polydecenes, fatty acid esters, pseudoceramides, pearlescent agents or other adjuvants according to the use envisaged.

The disclosed composition may also comprise up to 5% of pearlescent or opacifying agents well known in the state of the art, such as, for example, coated or non-coated titanium oxides, sodium or magnesium palmitates, sodium stearates, magnesium stearates, sodium hydroxystearates, and magnesium hydroxystearates, acylated derivatives comprising a fatty chain, such as monostearates or distearates, of ethylene glycol or of polyethylene glycol, ethers comprising fatty chains (for example, C₈-C₃₀ fatty chains), such as, for example, distearyl ether or 1-(hexadecyloxy)-2-octadecanol, cyclodextrins or fatty alcohols, such as cetyl, stearyl and behenyl alcohols.

Another embodiment of the invention is the use as shampoo of the composition defined above.

Another embodiment of the present invention is a process for washing keratinous substances comprising applying, to these substances, the disclosed composition, followed by rinsing the treated substances after an optional leave-in time.

The examples which follow are intended to illustrate the invention without, however, exhibiting a limiting nature.

### EXAMPLE 1

The following shampoo compositions were prepared:

| | Invention | | Comparative | |
|---|---|---|---|---|
| Composition | (A) | | (B) | |
| Sodium lauryl ether sulphate | 10 | g A.M. | 10 | g A.M. |
| (70/30 C₁₂/C₁₄) comprising | | | | |
| 2.2 mol of ethylene oxide | | | | |
| Sodium cocoylamphohydroxypropyl- | 3.2 | g A.M. | — | |
| sulphonate, as an aqueous solution | | | | |
| comprising 32% of active material, | | | | |
| provided under the name MIRANOL | | | | |
| CSE by Rhodia | | | | |
| Cocoamidopropyl betaine | — | | 3.2 | g A.M. |
| Hydroxyethylcellulose crosslinked with | 0.3 | g | 0.3 | g |
| epichlorohydrin and quaternized by | | | | |
| trimethylamine, sold under the name | | | | |
| JR400 by Amerchol | | | | |
| Monoisopropanolamide of | 2.5 | g | 2.5 | g |
| coconut acids | | | | |
| Preservatives | q.s. | | q.s. | |
| Citric acid or sodium hydroxide q.s. | pH 7 | | pH 7 | |
| Demineralized water q.s. for | 100 | g | 100 | g |

Shampooing was carried out by applying approximately 6 g of the composition A to a half-head of natural hair wetted beforehand. The shampoo was made to foam and was then rinsed copiously with water. The hair was dried with a hairdryer.

The same procedure as above was carried out with the comparative composition B on the other half-head.

The experts compared the half-heads in pairs. A panel of experts evaluated the appearance of the dried hair and recorded greater smoothness to the touch with the composition in accordance with the invention.

The hair treated with the composition A was significantly smoother than that treated with the composition B.

### EXAMPLE 2

The following shampoo compositions were prepared:

| | Invention | | Comparative | |
|---|---|---|---|---|
| Composition | (C) | | (D) | |
| Sodium lauryl ether sulphate | 14 | g A.M. | 14 | g A.M. |
| (70/30 C₁₂/C₁₄) | | | | |
| comprising 2.2 mol of ethylene oxide | | | | |
| Sodium cocoylamphohydroxypropyl- | 3 | g A.M. | — | |
| sulphonate, as an aqueous solution | | | | |
| comprising 32% of active material, | | | | |
| provided under the name | | | | |
| MIRANOL CSE by Rhodia | | | | |
| Sodium cocoamidoethyl(N- | — | | 3 | g A.M. |
| hydroxyethyl-N-carboxymethyl) | | | | |
| glycinate, sold under the | | | | |
| name MIRANOL C2M by Rhodia | | | | |
| Guar gum modified by 2,3-epoxy- | 0.18 | g | 0.18 | g |
| propyltrimethylammonium chloride, | | | | |
| sold under the name JAGUAR | | | | |
| C13 S by Rhodia | | | | |
| Polydimethylsiloxane with a viscosity | 2.5 | g | 2.5 | g |
| of 60 000 cSt, sold under | | | | |
| the name DC 200 | | | | |
| Fluid 60000 by Dow Corning | | | | |
| Mixture of 1-hexadecyloxy- | 2.5 | g | 2.5 | g |
| octadodecanol and of | | | | |
| cetyl alcohol | | | | |
| Preservatives | q.s. | | q.s. | |
| Fragrance | q.s. | | q.s. | |
| Citric acid or sodium hydroxide | pH 7.5 | | pH 7.5 | |
| (NaOH) q.s. | | | | |
| Demineralized water q.s. for | 100 | g | 100 | g |

Shampooing was carried out by applying approximately 6 g of the composition A to a half-head of natural hair wetted beforehand. The shampoo was made to foam and was then rinsed copiously with water. The hair was dried with a hairdryer.

The same procedure as above was carried out with the comparative composition B on the other half-head.

The experts compared the half-heads in pairs. A panel of experts evaluated the appearance of the dried hair and recorded greater body, setting and smoothness to the touch.

### EXAMPLES 3 AND 4

The following compositions were prepared:

| Composition | Example 3 | | Example 4 | |
|---|---|---|---|---|
| Sodium lauryl ether sulphate (70/30 | 11.2 | g A.M. | 16 | g A.M. |
| C₁₂/C₁₄) comprising | | | | |
| 2.2 mol of ethylene oxide | | | | |
| Sodium cocoylamphohydroxypropyl- | 3 | g A.M. | 2.7 | g A.M. |
| sulphonate, as an aqueous solution | | | | |
| comprising 32% of active material, | | | | |
| provided under the name | | | | |
| MIRANOL CSE by Rhodia | | | | |
| Hydroxyethylcellulose crosslinked by | 0.8 | g | — | |
| epichlorohydrin and quaternized by | | | | |
| trimethylamine, sold under the name | | | | |
| JR 400 by Amerchol | | | | |
| Guar gum modified by 2,3-epoxy- | — | | 0.4 | g |
| propyltrimethylammonium | | | | |
| chloride, sold under the name | | | | |
| JAGUAR C13 S by Rhodia | | | | |
| Glycol distearate | 2 | g | — | |
| Distearyl ether | — | | 1.5 | g |
| Behenyl alcohol | — | | 1.5 | g |
| Monoisopropanolamide of | 2.2 | g | — | |
| coconut acids | | | | |
| Crosslinked poly(acrylic acid) | 0.2 | g | 0.2 | g |
| Sodium chloride | — | | 1 | g |
| Citric acid | 3 | g | — | |
| Preservatives | q.s. | | q.s. | |
| Fragrance | q.s. | | q.s. | |
| Citric acid or sodium hydroxide | — | | pH 7.5 | |
| q.s. | | | | |
| Aqueous ammonia q.s. | pH 5.3 | | — | |
| Demineralized water q.s. for | 100 | g | 100 | g |

The hair washed with these compositions was smooth and easy to disentangle.

### EXAMPLES 5 AND 6

The following compositions were prepared:

| Composition | Example 5 | | Example 6 | |
|---|---|---|---|---|
| Sodium lauryl ether sulphate (70/30 | 1.5 | g A.M. | 1.1 | g A.M. |
| C₁₂/C₁₄) comprising | | | | |
| 2.2 mol of ethylene oxide | | | | |
| Sodium lauryl ether sulphate (70/30 | 6.3 | g A.M. | | |
| C₁₂/C₁₄) comprising | | | | |
| 3 mol of ethylene oxide | | | | |
| Ammonium lauryl ether sulphate | | | 8.5 | g A.M. |
| Sodium cocoylamphohydroxypropyl- | 3 | g A.M. | 3.2 | g A.M. |
| sulphonate, as an aqueous solution | | | | |
| comprising 32% of active material, | | | | |
| provided under the name | | | | |
| Miranol CSE by Rhodia | | | | |
| Sodium Cocoamidoethyl (N- | 0.8 | g AM | 0.5 | g AM |
| hydroxyethyl, N-carboxymethyl) | | | | |
| glycinate | | | | |
| (Miranol C2M by RHODIA) | | | | |
| Hydroxyethylcellulose crosslinked by | 0.3 | g | 0.4 | g |
| epichlorohydrin and quaternized by | | | | |
| trimethylamine, sold under the name | | | | |
| JR 400 by Amerchol | | | | |
| Glycol distearate | 2 | g | 1.5 | g |
| Monoethanolamide of coconut acids | 0.8 | g | — | |
| Crosslinked poly(acrylic acid) | 0.2 | g | 0.2 | g |
| Citric acid | Qs pH 6.7 | | 3 | g |
| Preservatives | q.s. | | q.s. | |
| Fragrance | q.s. | | q.s. | |
| Aqueous ammonia q.s. | | | pH 5.3 | |
| Demineralized water q.s. for | 100 | g | 100 | g |

What is claimed is:

1. A composition for washing keratinous substances, comprising, in a cosmetically acceptable aqueous medium,
at least one alkyl ether sulphate anionic surfactant, and
at least 2.5% of at least one alkylamphohydroxyalkylsulphonate amphoteric surfactant,
wherein the weight ratio of the at least one alkyl ether sulphate surfactant to the at least one alkylamphohydroxyalkylsulphonate amphoteric surfactant ranging from 2 to 20.

2. The composition according to claim 1, wherein the at least one alkyl ether sulphate anionic surfactant is a salt chosen from alkyl ether sulphates, alkylamido ether sulphates, and alkylaryl ether sulphates;
wherein an alkyl radical of said salt comprises from 8 to 24 carbon atoms, and
an aryl radical of said salt is chosen from a phenyl group and a benzyl group.

3. The composition according to claim 1, wherein the at least one alkyl ether sulphate anionic surfactant is chosen from C₁₀-C₁₄ alkyl ether sulphate salts.

4. The composition according to claim 1, wherein the at least one alkyl ether sulphate anionic surfactant is chosen from C₁₂-C₁₄ alkyl ether sulphate salts.

5. The composition according to claim 1, wherein the at least one alkylamphohydroxyalkylsulphonate amphoteric surfactant is chosen from a compound having the following formula (I): 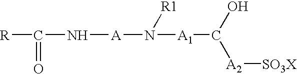
wherein:
R is chosen from saturated and unsaturated, linear and branched, hydrocarbonaceous radicals comprising from 5 to 29 carbon atoms,
R1 is a C₁-C₄ hydroxyalkyl radical,
A, A₁ and A₂, which are identical or different, are chosen from linear and branched, divalent C₁-C₁₀ alkylene radicals, and
X is chosen from a hydrogen atom, an inorganic cation, and an organic cation.

6. The composition according to claim 5, wherein R1 is a hydroxyethyl radical.

7. The composition according to claim 5, wherein A, A₁ and A₂, which are identical or different, are chosen from linear and branched, divalent C₁-C₃ alkylene radicals.

8. The composition according to claim 5, wherein R is chosen from alkyl and mono- and polyunsaturated alkenyl radicals comprising from 5 to 29 carbon atoms.

9. The composition according to claim 8, wherein R is chosen from alkyl and mono- and polyunsaturated alkenyl radicals comprising from 7 to 22 carbon atoms.

10. The composition according to claim 8, wherein R is chosen from alkyl and mono- and polyunsaturated alkenyl radicals comprising from 9 to 17 carbon atoms.

11. The composition according to claim 5, wherein A and A₂ are —CH₂CH₂—, and A₁ is —CH₂—.

12. The composition according to claim 5, wherein the inorganic cation and the organic cation are independently chosen from an alkali metal, an alkaline earth metal, NH₄⁺ ion, and ammonium ions resulting from basic amino acids or from aminoalcohols.

13. The composition according to claim 12, wherein the alkali metal cation is chosen from Na⁺ and K⁺ and the alkaline earth metal cation is chosen from Mg²⁺ and Ca²⁺.

14. The composition according to claim 1, wherein the at least one alkylamphohydroxyalkylsulphonate amphoteric surfactant is chosen from cocoylamphohydroxypropylsulphonate salts and palmitoylamphohydroxypropylsulphonate salts.

15. The composition according to claim 1, wherein the at least one alkyl ether sulphate anionic surfactant is present in the composition in an amount ranging from 2.5 to 50% by weight with respect to the total weight of the composition.

16. The composition according to claim 1, wherein the at least one alkyl ether sulphate anionic surfactant is present in the composition in an amount ranging from 5 to 40% by weight with respect to the total weight of the composition.

17. The composition according to claim 1, wherein the at least one alkylamphohydroxyalkylsulphonate amphoteric surfactant is present in the composition in an amount ranging from 2.5% to 30% by weight with respect to the total weight of the composition.

18. The composition according to claim 1, wherein the at least one alkylamphohydroxyalkylsulphonate amphoteric surfactant is present in the composition in an amount ranging from 3 and 15% by weight with respect to the total weight of the composition.

19. The composition according to claim 1, wherein the weight ratio of the at least one alkyl ether sulphate surfactant to the at least one alkylamphohydroxyalkylsulphonate amphoteric surfactant ranges from 2 to 10.

20. The composition according to claim 1, further comprising at least one additional surfactant chosen from nonionic, amphoteric, and zwitterionic surfactants other than the at least one alkylamphohydroxyalkylsulphonate amphoteric surfactant.

21. The composition according to claim 20, wherein the at least one additional surfactant is present in the composition in an amount ranging from 0.5 to 15% by weight with respect to the total weight of the composition.

22. The composition according to claim 1, wherein the total amount of surfactants present in the composition ranges from 5 to 50% by weight with respect to the total weight of the composition.

23. The composition according to claim 1, wherein the total amount of surfactants present in the composition ranges from 7 to 30% by weight with respect to the total weight of the composition.

24. The composition according to claim 1, further comprising at least one additional surfactant that is a cationic surfactant.

25. The composition according to claim 1, further comprising at least one silicone.

26. The composition according to claim 1, further comprising at least one polymer chosen from cationic and amphoteric polymers.

27. The composition according to claim 26, wherein the least one cationic polymer is chosen from cellulose ether derivatives comprising quaternary ammonium groups, non-cellulose polysaccharides, and cyclopolymers of methyidiallylamine or of diallyldimethylammonium.

28. The composition according to claim 27, wherein the non-cellulose polysaccharides are cationic guar gums.

29. The composition according to claim 1, further comprising at least one viscosity-regulating agent chosen from electrolytes and thickeners.

30. The composition according to claim 29, wherein the electrolytes are chosen from sodium chloride

31. The composition according to claim 29, wherein the thickeners are chosen from cellulose derivatives, guar gum, hydroxypropylated guar gums, scleroglucans, xanthan gum, and amphiphilic polymers comprising at least one fatty chain.

32. The composition according to claim 1, further comprising at least one adjuvant chosen from fragrances, preservatives, foam synergists, sequestering agents, foam stabilizers, propellants, colorants, antidandruff agents, ceramides, vitamins, provitamins, hydroxy acids, acidifying agents, basifying agents, vegetable, mineral oils, animal oils, synthetic oils, fatty acid esters, pseudoceramides, pearlescent agents, and opacifying agents.

33. The composition according to claim 32, wherein the synthetic oils are chosen from polyisobutenes and polydecenes.

34. The composition according to claim 32, wherein the pearlescent agents and opacifying agents are chosen from coated titanium oxides, non-coated titanium oxides, sodium palmitates, magnesium palmitates, sodium stearates, magnesium stearates, sodium hydroxystearates, magnesium hydroxystearates, acylated derivatives comprising a fatty chain, ethers comprising fatty chains, cyclodextrins, and fatty alcohols.

35. The composition according to claim 34, wherein the acylated derivatives comprising a fatty chain are chosen from monostearates and distearates of ethylene glycol and of polyethylene glycol.

36. The composition according to claim 34, wherein the ethers comprising fatty chains have C₈-C₃₀ fatty chains and are chosen from distearyl ether and 1-(hexadecyloxy)-2-octadecanol.

37. The composition according to claim 34, wherein the fatty alcohols are chosen from cetyl, stearyl, and behenyl alcohols.

38. A method for improving at least one of disentangling and smoothing of keratinous substances, comprising applying to hair a composition comprising, in a cosmetically acceptable aqueous medium,
at least one alkyl ether sulphate anionic surfactant, and
at least 2.5% of at least one alkylamphohydroxyalkylsulphonate amphoteric surfactant,
wherein the weight ratio of the at least one alkyl ether sulphate surfactant to the at least one alkylamphohydroxyalkylsulphonate amphoteric surfactant ranges from 2 to 20.

39. A shampoo composition comprising:
at least one alkyl ether sulphate anionic surfactant, and
at least 2.5% of at least one alkylamphohydroxyalkylsulphonate amphoteric surfactant,
wherein the weight ratio of the at least one alkyl ether sulphate surfactant to the at least one alkylamphohydroxyalkylsulphonate amphoteric surfactant ranges from 2 to 20.

40. A method for washing keratinous substances, comprising applying to the keratinous substances a composition comprising, in a cosmetically acceptable aqueous medium,
at least one alkyl ether sulphate anionic surfactant, and
at least 2.5% of at least one alkylamphohydroxyalkylsulphonate amphoteric surfactant, wherein the weight ratio of the at least one alkyl ether sulphate surfactant to the at least one alkylamphohydroxyalkylsulphonate amphoteric surfactant ranges from 2 to 20, and
rinsing the treated substances after an optional leave-in time.
